SEQUENCE LISTING (21 sequences)

SEQ ID NO: 1 - DNA, 18 bp, synthetic construct
  source 1..18: mol_type = other DNA
gggtggagtcgtgacgtg

SEQ ID NO: 2 - DNA, 13 bp, synthetic construct
  source 1..13: mol_type = other DNA
cctagtgatggag

SEQ ID NO: 3 - DNA, 4679 bp, synthetic construct
  source 1..4679: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 4 - DNA, 31 bp, synthetic construct
  source 1..31: mol_type = other DNA
cctagtgatggaggggtggagtcgtgacgtg

SEQ ID NO: 5 - DNA, 12 bp, synthetic construct
  source 1..12: mol_type = other DNA
aggggtggagtc

SEQ ID NO: 6 - DNA, 200 bp, synthetic construct
  source 1..200: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtga

SEQ ID NO: 7 - DNA, 205 bp, synthetic construct
  source 1..205: mol_type = other DNA
cccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctc

SEQ ID NO: 8 - DNA, 345 bp, synthetic construct
  source 1..345: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtga

SEQ ID NO: 9 - DNA, 305 bp, synthetic construct
  source 1..305: mol_type = other DNA
cccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 249 bp, synthetic construct
  source 1..249: mol_type = other DNA
ctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaag

SEQ ID NO: 11 - DNA, 32 bp, synthetic construct
  source 1..32: mol_type = other DNA
cccctagtgatgnaggggtggagtcntgacnt

SEQ ID NO: 12 - DNA, 27 bp, synthetic construct
  source 1..27: mol_type = other DNA
cctagtgatggaggggtggagtcntga

SEQ ID NO: 13 - DNA, 27 bp, synthetic construct
  source 1..27: mol_type = other DNA
tagtgatggaggggtggagtcgtgacg

SEQ ID NO: 14 - DNA, 36 bp, synthetic construct
  source 1..36: mol_type = other DNA
catggcgggttagtcattaactaccggcctcagtga

SEQ ID NO: 15 - DNA, 48 bp, synthetic construct
  source 1..48: mol_type = other DNA
aaggaacccctagtgatggagtcgtgacgtgaattacgtcatagggtt

SEQ ID NO: 16 - DNA, 38 bp, synthetic construct
  source 1..38: mol_type = other DNA
aggaacccctagtgatggaggggtggagtcgtgacgtg

SEQ ID NO: 17 - DNA, 41 bp, synthetic construct
  source 1..41: mol_type = other DNA
aacccctagtgatggagtcgtgacgtgaattacgtcatagg

SEQ ID NO: 18 - DNA, 32 bp, synthetic construct
  source 1..32: mol_type = other DNA
ctgaggccgcccgggcggaggggtggagtcgt

SEQ ID NO: 19 - DNA, 46 bp, synthetic construct
  source 1..46: mol_type = other DNA
atttctttcttatctagtttccatgctntagnncanggctangtag

SEQ ID NO: 20 - DNA, 32 bp, synthetic construct
  source 1..32: mol_type = other DNA
ttaatcattaactacaagcactaggggttcct

SEQ ID NO: 21 - DNA, 38 bp, synthetic construct
  source 1..38: mol_type = other DNA
acccctagtgatggaggggtggagtcgtgacgtgaatt

(19) United States
(12) Patent Application Publication
(10) Pub. No.: US 2024/0376494 A1
(43) Pub. Date: Nov. 14, 2024

(54) RECOMBINANT AAV CONSTRUCTS FOR INCREASED TRANSGENE EXPRESSION

(71) Applicant: Wisconsin Alumni Research Foundation, Madison, WI (US)

(72) Inventors: Kinjal Majumder, Madison, WI (US); Rhiannon Abrahams, Madison, WI (US)

(21) Appl. No.: 18/658,741

(22) Filed: May 8, 2024

Related U.S. Application Data
(60) Provisional application No. 63/500,725, filed on May 8, 2023.

(51) Int. Cl. C12N 15/86

(52) U.S. Cl. CPC C12N 15/86; C12N 2750/14122; C12N 2750/14143; C12N 2750/14151; C12N 2820/10

(57) ABSTRACT

Provided herein are constructs for producing recombinant adeno-associated virus (rAAV) vectors comprising rAAV genomes that can recombine to produce oligomerized rAAV genomes that persist longer in a cell than traditional rAAV genomes, and that comprise relatively large transgenes. The constructs comprise either a 5′ recombinant junction sequence, a 3′ recombinant junction sequence, or both, such that a recombination event will link the rAAV genomes together. Also provided are modified rAAV vectors comprising these constructs, methods for producing the vectors, and methods of using the vectors to deliver transgenes to a subject.

### CROSS-REFERENCE TO RELATED APPLICATIONS

This application claims the benefit of and priority to U.S. Provisional Application No. 63/500,725 filed on May 8, 2023, the content of which is incorporated by reference in its entirety.

### STATEMENT REGARDING FEDERALLY SPONSORED RESEARCH

This invention was made with government support under AI148511 awarded by the National Institutes of Health. The government has certain rights in the invention.

### SEQUENCE LISTING

The content of the electronic sequence listing (960296.04501.xml; Size: 25,439 bytes; and Date of Creation: May 8, 2024) is herein incorporated by reference in its entirety.

### BACKGROUND

Adeno-Associated Viruses (AAV) are modified to generate recombinant AAV (rAAV) gene therapy vectors by removing viral genes and replacing them with transgenes of interest. This could be used to develop therapeutic transgenes that can treat monogenic mutational diseases and also serve as a platform to perform genome editing. However, the use of AAV viruses and rAAV vectors is limited.

One limitation of rAAV gene therapy is that AAV genome copies are reduced over time, resulting in loss of transgene expression.

Also, rAAV vector are limited by the size of a DNA molecule that can be accommodated inside the capsid particle—the maximum size of DNA molecule in an rAAV gene therapy vector is approximately 5 kilobases. This is a major drawback in the current state of rAAV gene therapy vectors, preventing the use of large therapeutic transgenes and genome editing technologies from being delivered via rAAV.

Accordingly, there remains a need in the art for rAAV that can persist for a longer time period in the cell and accommodate larger DNA elements that may be used as therapeutic agents.

### SUMMARY

In a first aspect, a construct for producing a recombinant adeno-associated virus (rAAV) vector is provided herein. The construct comprises from 5′ to 3′: a 5′ inverted terminal repeat (ITR), a first recombinant junction sequence, a promoter operably linked to at least a portion of a transgene, and a 3′ ITR. The first recombinant junction sequence may comprise or consist of a KLF4 binding site comprising SEQ ID NO: 5. The first recombinant junction sequence may comprise SEQ ID NO: 6, SEQ ID NO: 8, a portion of SEQ ID NO: 6 or a portion of SEQ ID NO: 8.

The construct may further comprise a second recombinant junction sequence. The second recombinant junction sequence is between the transgene and the 3′ ITR and may overlap with the 3′ ITR. The second recombinant junction sequence may comprise at least the last 45 nucleotides of SEQ ID NO: 7 or may comprise SEQ ID NO: 9 or a portion thereof.

In another aspect, an rAAV vector comprising the construct described herein is provided.

In another aspect, a packaging cell transfected with the construct described herein, a packaging plasmid, and a helper plasmid to produce an rAAV vector comprising the construct is provided.

In another aspect, a composition comprising the rAAV vector comprising the construct described herein is provided.

In another aspect, methods of delivering a transgene to a cell are provided. These methods comprise contacting the cell with the compositions, rAAV vectors and constructs described herein.

In another aspect, methods of delivering a transgene to a subject are provided. The methods comprise administering the composition, rAAV vectors, and constructs described herein to the subject.

In another aspect, a system for expressing a transgene is provided. The system comprises a first construct for producing a first recombinant adeno-associated virus (rAAV) vector comprising, from 5′ to 3′: a 5′ inverted terminal repeat (ITR), a promoter operably linked to a 5′ portion of the transgene, a second recombinant junction sequence, and a 3′ ITR. The second recombinant junction sequence is between the transgene and the 3′ ITR and may overlap with the 3′ ITR. The system further comprises a second construct for producing a second rAAV vector comprising, from 5′ to 3′: the 5′ ITR, a first recombinant junction sequence, a 3′ portion of the transgene, a polyadenylation site, and the 3′ ITR.

In some embodiments, the first construct does not comprise the first recombinant junction sequence, and the second construct does not comprise the second recombinant junction sequence. The first recombinant junction sequence may comprises or consist of a KLF4 binding site comprising SEQ ID NO: 5. The first recombinant junction sequence comprises SEQ ID NO: 6 or a portion thereof.

The second recombinant junction sequence may comprise at least the last 45 nucleotides of SEQ ID NO: 7 or it may comprise SEQ ID NO: 9 or a portion thereof.

In some embodiments, the 5′ portion of the transgene and the 3′ portion of the transgene each comprise at least about 200 nucleotides, but may each comprise between about 200 nucleotides and about 4000 nucleotides.

In some embodiments, the first construct further comprises a splice donor site at the 3′ end of the 5′ portion of the transgene and the second construct further comprises a splice acceptor site and a branch site at the 5′ end of the 3′ portion of the transgene.

A recombinant AAV (rAAV) vector comprising the first construct or the second construct described herein are also provided.

A packaging cell transfected with the first construct or the second construct described herein, a packaging plasmid, and a helper plasmid to produce a rAAV vector comprising the first construct or the second construct is also provided.

A composition comprising a first rAAV particle comprising the first construct and a second rAAV particle comprising the second construct described herein is also provided. The compositions and rAAV vectors comprising both the first and second construct may be used to deliver a transgene to a cell or to a subject by contacting cells or administering compositions comprising rAAV comprising both the first and second construct. The system provided then allows for recombination between the two recombinant AAV in a cell in a particular orientation to allow the 5′ portion of the transgene and the 3′ portion of the transgene to be transcribed as a single transcript and allow for translation of the recombined transcript to allow for expression of the transgene.

In another aspect, a system for expressing a transgene is provided. The system comprises: a first construct for producing a first recombinant adeno-associated virus (rAAV) vector, a second construct for producing a second rAAV vector, and an intervening construct for producing an intervening rAAV vector. The first construct comprising, from 5′ to 3′: a 5′ ITR, a promoter operably linked to a 5′ portion of the transgene, a first 3′ recombinant junction sequence, and a 3′ ITR. The second construct comprising, from 5′ to 3′: the 5′ ITR, a second 5′ recombinant junction sequence, a 3′ portion of the transgene, a polyadenylation site, and the 3′ ITR. The intervening construct comprising, from 5′ to 3′: the 5′ ITR, a first 5′ recombinant junction sequence, an internal portion of the transgene, a second 3′ recombinant junction sequence, and the 3′ ITR. The first 5′ recombinant junction sequence and the second 5′ recombination sequence are different. The first 3′ recombinant junction sequence and the second 3′ recombinant junction sequence are different. The first 5′ recombinant junction sequence and the first 3′ recombinant junction sequence are such that when recombined, the 5′ portion of the transgene is in frame with the internal portion of the transgene. The second 3′ recombinant junction sequence and the second 5′ recombinant junction sequence are such that when recombined, the internal portion of the transgene is in frame with the 3′ portion of the transgene.

In some embodiments, the first construct does not comprise a 5′ recombinant junction sequence, and the second construct does not comprise a 3′ recombinant junction sequence.

In some embodiments, the second construct further comprises a KLF4 binding site in the 5′ recombinant junction sequence, the intervening construct further comprises the KLF4 binding site in the 5′ recombinant junction sequence, and the first construct does not comprise a KLF4 binding site.

In some embodiments, the 5′ portion of the transgene, the 3′ portion of the transgene, and the internal portion of the transgene each comprise between about 200 nucleotides and about 4000 nucleotides.

In some embodiments, the first construct further comprises a splice donor site at the 3′ end of the 5′ portion of the transgene; the second construct further comprises a splice acceptor site and a branch site at the 5′ end of the 3′ portion of the transgene; and the intervening construct further comprises the splice donor site at the 3′ end of the internal portion of the transgene, and the splice acceptor site and the branch site at the 5′ end of the internal portion of the transgene.

### BRIEF DESCRIPTION OF THE DRAWINGS

FIG. 1 is a schematic illustrating the standard use of AAV2 genomes to engineer recombinant AAV2 (rAAV) vectors.

FIG. 2 is a schematic illustrating the current state of the art using rAAV genomes to reconstitute larger transgenes via splice donor and acceptor sites.

FIG. 3 is a schematic illustrating formation of AAV2 monomers into concatemers.

FIG. 4 is a schematic of AAV2 rearrangement leading to concatemer formation and the identification of recombined junctions through Sanger sequencing, with PCR primers indicated.

FIG. 5 is a schematic of the AAV2 genome showing the location of the ITR elements and KLF4 binding region. The individual Topo clones that were sequenced are represented by red arrows with the aligned sequence represented in red blocks.

FIGS. 6A-6C. FIG. 6A is a schematic of the rearrangement between successive AAV2 genomes showing the location of qPCR primers. FIG. 6B is a time course of AAV2 rearrangement in HEK293T cells. FIG. 6C demonstrates reduced rearrangement in the presence of PARP inhibitor, Olaparib.

FIG. 7 is a schematic illustrating insertion of AAV2 rearrangement sequences into rAAV vectors to reconstitute rAAV transgenes expressing fluorescent reporter (GFP).

FIG. 8 is a schematic illustrating an AAV recombination reference. The sequence depicts the AAV recombined junction identified by TOPO-cloning. The intervening region which is lost during recombination is labelled as “Rearrangement”.

FIG. 9 is a schematic illustrating recombined junction sequences aligned to an AAV recombined junction reference. The arrows indicate successful alignments to the reference in unique reads. The most abundant rearrangement independently identified by TOPO-cloning is the arrow labeled “***”. Successful sequences identified are indicated in solid regions of the *** arrow. Empty reads in the arrows are indicated by the open regions of the arrows. These regions represent “Rearrangements”.

FIG. 10 is a schematic illustrating an AAV2 dimer and regions of the AAV5′ end that are common and unique to AAV and traditional rAAVs.

### DETAILED DESCRIPTION

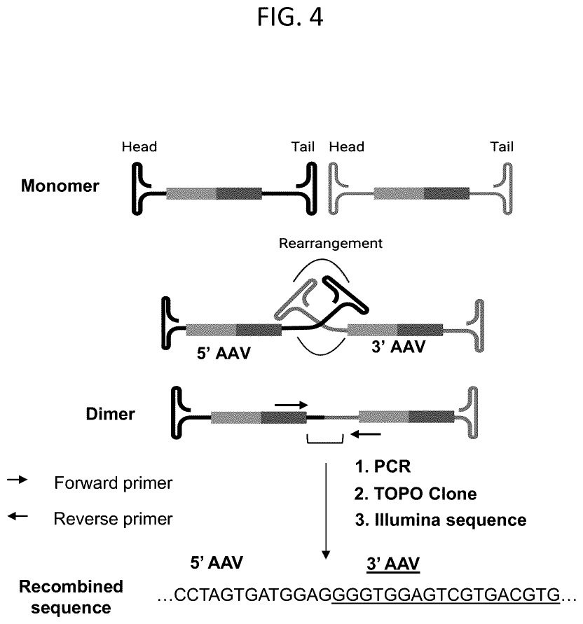

The present disclosure provides systems and methods for producing recombinant adeno-associated virus (rAAV) genomes that may concatemerized for increased persistence and/or may be used to reconstitute segments of large transgenes for expression within a cell. The inventors have discovered that AAV2 concatemer formation involves recombinant junction sequences at each end of an AAV2 genome monomer. A recombinant junction sequence at the 3′ end of one monomer can recombine with a recombinant junction sequence at the 5′ end of another monomer, resulting in two monomers linked together to form a dimer comprising the genes expressed in both monomers. See, e.g. FIG. 4. The inventors have also discovered that a Kruppel-like factor 4 (KLF4) binding site in the recombinant junction sequence at the 5′ end of a monomer is required for the recombination event to occur. In rAAV vectors currently used in the art, these sequences have been removed from the genomes in order to accommodate larger transgene sequences. As described herein, the inventors have engineered rAAV vectors that include portions of the recombination junction sequences, including the KLF4 binding site, so that concatemerization of the genomes can occur. These rAAV vectors may be used to increase the persistence of the genomes in a cell, as well as increase the size of transgenes that may be expressed in a cell, which enhances the availability of large transgenes as therapeutics in rAAV systems.

### Constructs and Systems:

### Constructs for Increasing Concatemer Formation

In a first aspect, the present disclosure provides a construct for producing a recombinant adeno-associated virus (rAAV) vector. The rAAV produced from the construct may include a transgene and allow for expression of the transgene in cells contacted with the rAAV. The construct comprises from 5′ to 3′: a 5′ inverted terminal repeat (ITR), a first recombinant junction sequence, a promoter operably linked to the transgene, a polyadenylation site, and a 3′ ITR. The first recombinant junction sequence may comprise a KLF4 binding site, and the KLF4 binding site may be at a 5′ region of the first recombinant junction sequence. The construct may further comprise a second recombinant junction sequence, wherein the second recombinant junction sequence is position between the transgene and the 3′ ITR and may partially overlap with the 3′ ITR. The construct may also include a polyadenylation site between the transgene and the 3′ ITR.

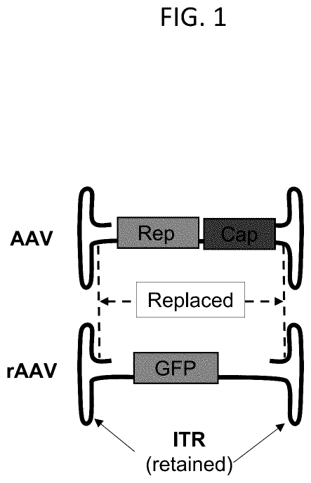

Adeno associated viruses (AAV) are non-pathogenic viruses that belong to the genus Dependoparvovirus. AAV are small, nonenveloped viruses that have a linear single-stranded DNA genome that is approximately 4.7 kilobases (kb) in size. Their genomes encode two distinct sets of proteins: the non-structural replication (Rep) proteins, and the capsid (Cap) proteins that form the structure into which the genome is packaged (FIG. 1). AAV viruses are replication defective, meaning that the production of AAV virus requires coinfection with helper virus(es). AAV offer several advantages for use as gene therapy vectors: AAV-based gene therapy vectors cause a very mild immune response, can infect both dividing and quiescent cells, and generally persist in an extrachromosomal state without integrating into the genome of the host cell.

The terms “rAAV vector”, “viral vector”, “AAV vector”, and “virus particle” are used interchangeably herein to describe a virion that is used to deliver genetic material (e.g., the constructs or portions thereof described herein) into a cell. An rAAV particle consists of a nucleic acid, such as the constructs disclosed herein, surrounded by a protective protein coat called a capsid.

As used herein, the term “construct” refers to a recombinant polynucleotide. As used herein, the terms “recombinant polynucleotide” and “recombinant nucleic acid” refer to nucleic acid sequences having an artificial combination of two or more sequences that do not naturally occur together or are from different sources (natural or synthetic). For example, the constructs described herein comprise at least a portion of the coding region of a transgene of interest operably linked to a promoter that (1) is associated with another gene found within the same genome, (2) from the genome of a different species, or (3) is synthetic. Constructs can be generated using conventional recombinant DNA methods. The constructs described herein may be single stranded polynucleotides packaged in a rAAV or may be a plasmid or other double stranded polynucleotide that may be transfected with a packaging and/or helper plasmid to allow for rAAV packaging and production. In the rAAV vectors and the constructs described herein the Rep/Cap genes and their regulatory sequences have been replaced with a transgene in the AAV genome, as depicted in FIG. 1 (where GFP is the transgene).

The constructs described herein also comprise inverted terminal repeats flanking the transgene at both the 5′ and 3′ ends. The constructs may include further sequences to allow for replication of the plasmid or selection of cells comprising the construct. These further sequences may be in a portion of the construct not flanked by the 5′ and 3′ ITRs, such that those further sequences are not packaged into the rAAV. “Inverted terminal repeats (ITRs)” are palindromic G-C-rich inverted repeats found on each end of the single stranded AAV genome, which self-base-pair to form unique AAV genome structures. ITRs contain several cis-acting elements that are involved in the initiation of viral DNA replication, as well as binding motifs for cellular transcription factors. Thus, the inclusion of ITRs in the constructs allows the constructs to be incorporated into an AAV particle and replicated for viral production.

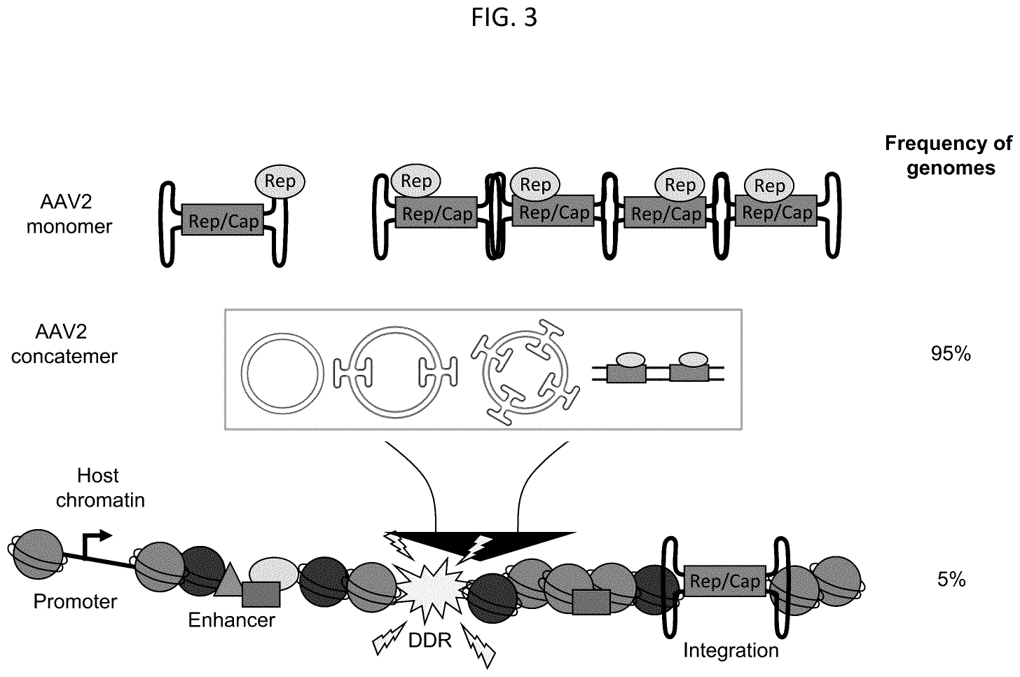

A wild-type AAV vector comprises an encapsulated genome, in which the genome is flanked at the 5′ and 3′ ends by inverted terminal repeats (ITRs). When not encapsulated, a single AAV genome (or monomer) is capable of recombining with another AAV monomer, one at its 5′ end and the other at its 3′ end, to form a dimer in which large portions of the ITRs between them are removed. Multiple AAV monomers can be joined in this way, head to tail, to form multimers. This process, called concatemerization, can yield large DNA molecules containing multiple identical AAV monomers linked in a series (FIG. 3).

The term “recombinant junction sequence”, as used herein, refers to an AAV sequence that aids in a recombination event between two AAV monomers, such that the two AAV monomers concatemerized.

A “first recombinant junction sequence” or “5′ recombinant junction sequence” is a recombinant junction sequence located 5′ of the transgene. In embodiments, the 5′ recombinant junction sequence is between the 5′ ITR and the transgene. In other embodiments, the 5′ recombinant junction sequence includes at least a portion of the 5′ ITR. The 5′ recombinant junction sequence may comprise or consist of a KLF4 binding site. The KLF4 binding site comprises the sequence of aggggtggagtc (SEQ ID NO: 5). KLF4 is a member of the KLF family of zinc finger transcription factors. KLF4 has been shown to bind to, and be regulated by, poly(ADP-ribose) polymerase 1 (PARP1), which plays a role in repair of single-stranded DNA breaks. As shown in the Examples, when the KLF4 binding site is removed from the 5′ recombinant junction sequence, recombination does not occur. The 5′ recombinant junction sequence may comprise a sequence having substantial identity to SEQ ID NO: 6 or portions thereof, including the KLF4 binding site. The 5′ recombinant junction sequence may comprise a sequence having substantial identity to SEQ ID NO: 8 or portions thereof. The 5′ recombinant junction sequence may comprise a sequence having substantial identity to SEQ ID NO: 8 or portions thereof, including the KLF4 binding site. The portions of SEQ ID NO: 6 or SEQ ID NO: 8 included as the 5′ or first recombinant junction sequence may be at least 25 nucleotides, at least 50 nucleotides, at least 75 nucleotides, at least 100 nucleotides, at least 125 nucleotides or any portion up to the full-length sequences provided. Suitably, the portions include the KLF4 binding site. The portions may be the 5′ portion of the sequence, the 3′ portion of the sequence or a middle portion of the sequence.

A “second recombinant junction sequence” or “3′ recombinant junction sequence” is a recombinant junction sequence located 3′ of the transgene. In embodiments, the 3′ recombinant junction sequence partially overlaps with the 3′ ITR. The 3′ recombinant junction sequence may comprise a sequence having substantial identity to SEQ ID NO: 7 or portions thereof. The 3′ recombinant junction sequence may comprise at least the last 45 nucleotides of a sequence having substantial identity to SEQ ID NO: 7. The 3′ recombinant junction sequence may comprise at least the first 50 nucleotides, 100 nucleotides, 150 nucleotides, or 200 nucleotides of a sequence having substantial identity to SEQ ID NO: 7. The 3′ recombinant junction sequence may comprise a sequence having substantial identity to SEQ ID NO: 9 or portions thereof. The portions of SEQ ID NO: 7 or SEQ ID NO: 9 included as the 3′ or second recombinant junction sequence may be at least 25 nucleotides, at least 50 nucleotides, at least 75 nucleotides, at least 100 nucleotides, at least 125 nucleotides or any portion up to the full-length sequences provided. The portions may be the 5′ portion of the sequence, the 3′ portion of the sequence or a middle portion of the sequence.

As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

A “polyadenylation site” is a site within a gene or transgene, typically near the 3′ end, at which, transcription of the gene stops and a poly(A) tail is added to the mRNA transcript. In eukaryotes, polyadenylation is part of the process that produces mature mRNA for translation. The poly(A) tail is important for nuclear export, translation, and stability of the mRNA.

As used herein, the term “nucleic acid” or “polynucleotide” refers to deoxyribonucleic acid (DNA), ribonucleic acid (RNA) and DNA/RNA hybrids. Polynucleotides may be single-stranded or double-stranded. Nucleic acids include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA (+)), minus strand RNA (RNA (−)), synthetic RNA, genomic DNA (gDNA), PCR amplified DNA, complementary DNA (cDNA), synthetic DNA, or recombinant DNA.

As used herein, the terms “protein” or “polypeptide” or “peptide” are used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

As used herein, the term “transgene” or “transgene of interest” refers to a gene or genetic material that one wishes to transfer into an organism or a cell thereof. A transgene may encode any protein or functional RNA of interest. Suitable transgenes include those that encode a therapeutic product. For example, the transgene may encode a protein that is lacking due to a genetic disorder or may encode a small interfering RNA (siRNA) that downregulates the expression of a protein that is overexpressed or ectopically expressed due to a genetic disorder. Any suitable transgene for use in gene therapy is contemplated for use in the present disclosure.

### Constructs for Increasing Concatemer Formation and Expressing Large Transgenes

In the Examples, the inventors identified a recombined junction (SEQ ID NO: 4) in a dimerized AAV2, having a 5′ sequence (SEQ ID NO: 1) provided from an AAV2 monomer on its 3′ end, and a 3′ sequence (SEQ ID NO: 2) provided by an AAV2 monomer on its 5′ end. A recombination event joining the 5′ sequence and 3′ sequence yields a dimerized AAV2 incorporating the genes within each monomer. Similarly, when two rAAV monomers, each expressing a portion of a large transgene are recombined at the junction, the resulting dimerized rAAV includes the two transgene segments, allowing for expression of larger transgenes than could be accommodated in a single rAAV monomer. Therefore, the system can yield transgenes larger than 5 kb.

Accordingly, in a second aspect, provided herein is a system for expressing a transgene. The system comprising two constructs for producing a first and a second recombinant adeno-associated virus (rAAV) vector. The constructs are designed to allow for dimerization between the two rAAVs to allow for expression of a large transgene via dimerization of the rAAV vectors in an ordered fashion. The first construct comprises, from 5′ to 3′: a 5′ inverted terminal repeat (ITR), a promoter operably linked to a 5′ portion of the transgene, a second recombinant junction sequence, and a 3′ ITR, wherein the second recombinant junction sequence is positioned between the transgene and the 3′ ITR. The second construct for producing the second rAAV vector comprises, from 5′ to 3′: the 5′ ITR, a first recombinant junction sequence, a 3′ portion of the transgene, a polyadenylation site, and the 3′ ITR. In embodiments, the first construct does not comprise the second recombinant junction sequence, and the second construct does not comprise the first recombinant junction sequence. The positioning and/or lack of recombination junction sequences in each construct allows for preferential ordered dimerization of the first rAAV to the second rAAV to allow the promoter to drive expression of the 5′ portion of the transgene, the 3′ portion of the transgene and polyadenylation site to allow for effective transcription and translation of the full-length transgene by the dimer in a cell.

In embodiments, the first recombinant junction sequence is between the 5′ ITR and the 3′ portion of the transgene in the second construct or second rAAV. In other embodiments, the first recombinant junction sequence includes at least a portion of the 5′ ITR. The first recombinant junction sequence may comprise or consist of a KLF4 binding site (SEQ ID NO: 5). The first recombinant junction sequence may comprise a sequence having substantial identity to SEQ ID NO: 6 or portions thereof, including the KLF4 binding site. The first recombinant junction sequence may comprise a sequence having substantial identity to SEQ ID NO: 8 or portions thereof. The first recombinant junction sequence may comprise a sequence having substantial identity to SEQ ID NO: 8 or portions thereof, including the KLF4 binding site. The portions of SEQ ID NO: 6 or SEQ ID NO: 8 included as the 5′ or first recombinant junction sequence may be at least 25 nucleotides, at least 50 nucleotides, at least 75 nucleotides, at least 100 nucleotides, at least 125 nucleotides or any portion up to the full-length sequences provided. Suitably, the portions include the KLF4 binding site. The portions may be the 5′ portion of the sequence, the 3′ portion of the sequence or a middle portion of the sequence.

In embodiments, the second recombinant junction sequence is located 3′ of the 5′ portion of the transgene in the first construct or first rAAV. In embodiments, the second recombinant junction sequence is positioned between the transgene and the 3′ ITR and may overlap with the 3′ ITR. The second recombinant junction sequence may comprise a sequence having substantial identity to SEQ ID NO: 7 or portions thereof. The second recombinant junction sequence may comprise at least the last 45 nucleotides of a sequence having substantial identity to SEQ ID NO: 7. The second recombinant junction sequence may comprise at least the first 50 nucleotides, 100 nucleotides, 150 nucleotides, or 200 nucleotides of a sequence having substantial identity to SEQ ID NO: 7. The second recombinant junction sequence may comprise a sequence having substantial identity to SEQ ID NO: 9 or portions thereof. The portions of SEQ ID NO: 7 or SEQ ID NO: 9 included as the 3′ or second recombinant junction sequence may be at least 25 nucleotides, at least 50 nucleotides, at least 75 nucleotides, at least 100 nucleotides, at least 125 nucleotides or any portion up to the full-length sequences provided. The portions may be the 5′ portion of the sequence, the 3′ portion of the sequence or a middle portion of the sequence.

The 5′ portion of the transgene and the 3′ portion of the transgene may comprise at least about 200 nucleotides. The 5′ portion of the transgene and the 3′ portion of the transgene may each comprise between about 200 and about 4000 nucleotides (nt), and lengths and ranges in between, e.g. about 300 nt, about 400 nt, about 500 nt, about 600 nt, about 700 nt, about 800 nt, about 900 nt, about 1 kb, about 1.5 kb, about 2 kb, about 2.5 kb, about 3 kb, about 3.5 kb, about 4 kb, about 4.5 kb, etc. Those of skill in the art will appreciate that the split in the transgene between the two constructs need not be even and can be selected to cause minimal effect on protein function. The position of the split in the transgene sequence may be selected to be in a portion of the transgene that has less structure or is unstructured, e.g., within a loop of the protein structure produced by expression of the transgene such that a recombination event that maintains the open reading frame but includes extra amino acids or results in a change in the amino acid sequence of the protein does not destroy protein function while allowing for the sequences needed to allow concatemerization.

The constructs of the system are configured in such a way that concatemerization between an AAV genome comprising the first construct and an AAV genome comprising the second construct allows for transcription of a full transgene, translation to produce a protein and production of a functional protein. The constructs may be configured such that, when recombined, the 3′ portion of the transgene is in frame with the 5′ portion of the transgene.

The first construct may further comprise a splice donor site at the 3′ end of the 5′ portion of the transgene. The second construct may further comprise a splice acceptor site at the 5′ end of the 3′ portion of the transgene. The 5′ end of the 3′ portion of the transgene may further comprise a branch site. RNA splicing is a process in which a pre-mRNA transcript is transformed into mature mRNA. Introns (non-coding regions of RNA) are removed, and the exons (coding regions) at each end of an intron are spliced together. In the constructs described herein, artificial splice donor and splice acceptor sites may be added to increase removal of non-transgene or AAV nucleotides remaining within the mRNA sequence after recombination.

The splice donor site comprises an almost invariant GU sequence at the 5′ end of the intron, which exists within a larger, less highly conserved region. The splice acceptor site comprises an almost invariant AG sequence at the 3′ end of the intron. Upstream from the AG there is a region containing a high level of pyrimidines (C and U) or polypyrimidine tract. Upstream from the polypyrimidine tract there is a branch site, which includes the adenine nucleotide that is involved in lariat formation. The sequences that can be used as splice donor and splice acceptor sites are variable but highly conserved. Splice donor, splice acceptor, and branch sites for the constructs described herein can be readily determined by one of skill in the art.

In a third aspect, provided herein is a system for expressing a transgene, the system comprising three or more constructs for expressing a larger transgene. The system may comprise a first construct for producing a first recombinant adeno-associated virus (rAAV) vector. The first construct comprising, from 5′ to 3′: a 5′ ITR, a promoter operably linked to a 5′ portion of the transgene, a 3′ recombinant junction sequence, and a 3′ ITR. As above, the first 3′ recombinant junction sequence is positioned between the transgene and the 3′ ITR and may overlap with the 3′ ITR. The second construct for producing a second rAAV vector, may comprise, from 5′ to 3′: the 5′ ITR, a second 5′ recombinant junction sequence, a 3′ portion of the transgene, a polyadenylation site, and the 3′ ITR. One or more intervening constructs for producing at least one intervening rAAV vector is also included. The intervening construct comprises, from 5′ to 3′: the 5′ ITR, a first 5′ recombinant junction sequence, an internal portion of the transgene, a second 3′ recombinant junction sequence, and the 3′ ITR. The second 3′ recombinant junction sequence is between the internal portion of the transgene and the 3′ ITR and may overlap with the 3′ ITR. In this system, the first 5′ recombinant junction sequence and the second 5′ recombination sequence may be different and the first 3′ recombinant junction sequence and the second 3′ recombinant junction sequence may be different to allow for coordinated recombination between compatible recombinant junction sequences to provide for an ordered recombination such that the transgene pieces are positioned correctly to allow for expression of the gene after recombination and concatemerization of the rAAV in a cell. The first 5′ recombinant junction sequence and the first 3′ recombinant junction sequence are configured such that when recombined the 5′ portion of the transgene is in frame with the internal portion of the transgene. The second 3′ recombinant junction sequence and the second 5′ recombinant junction sequence are configured such that when recombined, the internal portion of the transgene is in frame with the 3′ portion of the transgene. The constructs of the system are configured in such a way that concatemerization between the first construct, the intervening construct, and the second construct allows for transcription of a full transgene. In embodiments, the first construct does not comprise a 5′ recombinant junction sequence, and the second construct does not comprise a 3′ recombinant junction sequence.

In embodiments, the first 5′ recombinant junction sequence is between the 5′ ITR and the internal portion of the transgene. In other embodiments, the first 5′ recombinant junction sequence includes at least a portion of the 5′ ITR. The first 5′ recombinant junction sequence may comprise or consist of a KLF4 binding site (SEQ ID NO: 5). The first 5′ recombinant junction sequence may comprise a sequence having substantial identity to SEQ ID NO: 6 or portions thereof, including the KLF4 binding site. The first 5′ recombinant junction sequence may comprise a sequence having substantial identity to SEQ ID NO: 8 or portions thereof. The first 5′ recombinant junction sequence may comprise a sequence having substantial identity to SEQ ID NO: 8 or portions thereof, including the KLF4 binding site. Portions thereof are as defined above.

In embodiments, the first 3′ recombinant junction sequence is located 3′ of the 5′ portion of the transgene. In embodiments, the first 3′ recombinant junction sequence may partially overlap with the 3′ ITR. The first 3′ recombinant junction sequence may comprise a sequence having substantial identity to SEQ ID NO: 7 or portions thereof. The first 3′ recombinant junction sequence may comprise at least the last 45 nucleotides of a sequence having substantial identity to SEQ ID NO: 7. The first 3′ recombinant junction sequence may comprise at least the first 50 nucleotides, 100 nucleotides, 150 nucleotides, or 200 nucleotides of a sequence having substantial identity to SEQ ID NO: 7. The first 3′ recombinant junction sequence may comprise a sequence having substantial identity to SEQ ID NO: 9 or portions thereof. Portions thereof are as defined above.

In embodiments, the second 5′ recombinant junction sequence is between the 5′ ITR and the 3′ portion of the transgene. In other embodiments, the second 5′ recombinant junction sequence may include at least a portion of the 5′ ITR. The second 5′ recombinant junction sequence may comprise or consist of a KLF4 binding site (SEQ ID NO: 5). The second 5′ recombinant junction sequence may comprise a sequence having substantial identity to SEQ ID NO: 6 or portions thereof, including the KLF4 binding site. The second 5′ recombinant junction sequence may comprise a sequence having substantial identity to SEQ ID NO: 8 or portions thereof. The second 5′ recombinant junction sequence may comprise a sequence having substantial identity to SEQ ID NO: 8 or portions thereof, including the KLF4 binding site. Portions thereof are as defined above.

In embodiments, the second 3′ recombinant junction sequence is located 3′ of the internal portion of the transgene. In embodiments, the second 3′ recombinant junction sequence may at least partially overlap with the 3′ ITR. The second 3′ recombinant junction sequence may comprise a sequence having substantial identity to SEQ ID NO: 7 or portions thereof. The second 3′ recombinant junction sequence may comprise at least the last 45 nucleotides of a sequence having substantial identity to SEQ ID NO: 7. The second 3′ recombinant junction sequence may comprise at least the first 50 nucleotides, 100 nucleotides, 150 nucleotides, or 200 nucleotides of a sequence having substantial identity to SEQ ID NO: 7. The second 3′ recombinant junction sequence may comprise a sequence having substantial identity to SEQ ID NO: 9 or portions thereof. Portions thereof are as defined above.

In order to increase the directional arrangement of the constructs, the recombinant junction sequences that recombine may be different. The recombinant junction sequences provided herein are from AAV2. However, recombinant junction sequences from other serotypes may be used. Other AAV serotypes include AAV1, AAV4, AAV5, AAV6, AAV7, AAV8, AAV9.

The 5′ portion of the transgene, the internal portion of the transgene, and the 3′ portion of the transgene may each comprise at least about 200 nucleotides. Each portion of the transgene may comprise between about 200 and about 4000 nucleotides (nt), and lengths and ranges in between, e.g. about 300 nt, about 400 nt, about 500 nt, about 600 nt, about 700 nt, about 800 nt, about 900 nt, about 1 kb, about 1.5 kb, about 2 kb, about 2.5 kb, about 3 kb, about 3.5 kb, about 4 kb, about 4.5 kb, etc. Those of skill in the art will appreciate that the split in the transgene between the two constructs need not be even and can be selected to cause minimal effect on protein function. The position of the split in the transgene sequence may be selected to be in a portion of the transgene that has less structure or is unstructured, e.g., within a loop of the protein structure produced by expression of the transgene such that a recombination event that maintains the open reading frame but includes extra amino acids or results in a change in the amino acid sequence of the protein does not destroy protein function while allowing for the sequences needed to allow concatemerization.

The first construct may comprise a splice donor site at the 3′ end of the 5′ portion of the transgene; the second construct may comprise a splice acceptor site and a branch site at the 5′ end of the 3′ portion of the transgene; and the intervening construct may comprise both the splice donor site at the 3′ end of the internal portion of the transgene, and the splice acceptor site and the branch site at the 5′ end of the internal portion of the transgene. As described above, these splice donors and acceptors may allow for removal of intervening sequences left from the recombination of the three or more rAAVs and to allow for effective translation of the protein encoded by the split transgene.

### Viral Particles, Packaging Cell Lines, Host Cells, and Compositions:

In a fourth aspect, provided herein is an rAAV vector comprising any of the constructs described herein.

In a fifth aspect, provided herein is a packaging cell transfected with any of the constructs described herein, a packaging plasmid, and a helper plasmid. The terms “packaging cell” and “packaging cell line” refer to a cell line that provides all the proteins necessary for AAV virus production and maturation. Suitable packaging cell lines for use with the constructs described herein include, without limitation, mammalian cells and human cell lines. For example, suitable cell lines include, but are not limited to, HEK293T cells and HEK293 cell variants. The packaging cell line should be selected with the method of viral production in mind. For example, cells that have strong adhesion properties should be selected for growth in culture plates, whereas cells lacking adhesion properties should be selected for growth in suspension culture. The packaging cell line comprises the complement of any genes that have been functionally deleted in the virus particle used to produce the virus, such as in a helper plasmid and packaging plasmid, allowing replication incompetent viral particles to be produced.

A “plasmid” is a small circular DNA molecule that can replicate independently from chromosomal DNA. In nature, plasmids are commonly found in bacteria, and artificial plasmids are widely used as vectors in molecular cloning. When referring to a nucleic acid molecule alone, the terms “plasmid” and “vector” are used interchangeably herein to describe a nucleic acid molecule capable of transporting another nucleic acid to which it is linked. In contrast, and as discussed above, the term “viral vector”, “AAV vector”, or “rAAV vector” is used to describe a virus particle that is used to deliver genetic material into cells. The construct for producing the rAAV vector may be provided in a plasmid.

The term “packaging plasmid” refers to a plasmid that encodes components of the AAV proteins. For rAAV production, the packaging plasmid may encode the AAV genes Rep and Cap. The term “helper plasmid” refers to a plasmid that encodes adenovirus helper functions. Proteins encoded by all three plasmids in the packaging cell are required for rAAV production and AAV replication, as is well known in the art.

In a sixth aspect, the present disclosure provides host cells transduced with the constructs or rAAV described herein. As used herein, the term “host cell” refers to any eukaryotic cell that has been transduced with an rAAV vector containing a construct described herein. This term also includes cells that have been genetically engineered such that a construct of the present disclosure is integrated into its genome.

Method for Producing rAAV Vectors:

In a seventh aspect, the present disclosure provides methods for producing a composition comprising the rAAV vectors described herein. The methods comprise: (a) transfecting a packaging cell with a plasmid comprising any of the constructs described herein, a packaging plasmid, and a helper plasmid; (b) collecting the supernatant and the cells from culture; and (c) isolating virus particles from the supernatant and cells.

Virus particles can be isolated from the supernatant and/or from lysed cells by methods known and understood in the art. Suitable methods for isolating virus particles from cell culture include, but are not limited to, cesium chloride density gradient centrifugation and affinity purification (e.g., using a porous matrix modified to retain the virus).

The methods may further comprise concentrating the virus particles. Suitable methods for concentrating particles include, but are not limited to, ultracentrifugation and dialysis.

The methods may further comprise dialyzing the supernatant. For some applications, it may be advantageous to replace the cell culture media present in the supernatant with a solution that is better for long-term storage. Suitable solutions for storage include, but are not limited to, phosphate-buffered saline (PBS), PBS with plutonic acid, saline adjusted to pH 7-7.4 with or without pluronic acid (0.001-0.01%), and Ringer's lactate solution. However, any biocompatible, osmotically balanced, neutral pH fluid should be suitable for storage.

The terms “transduced,” “transfected,” and “transformed” all refer to processes by which an exogenous nucleic acid is introduced into a host cell. The term “transduced” specifically refers to the process by which a virus transfers a nucleic acid into a host cell. Plasmids may be used to transfect the construct into a host cell for AAV production along with the helper viruses. For a detailed description of viral production methods, see Ayuso et al. (Gene Ther 17 (4): 503-10, 2010), which is hereby incorporated by reference in its entirety. Other suitable methods for producing AAV virus particles are well known and understood in the art.

In an eighth aspect, provided herein are compositions comprising the constructs or rAAVs described herein. In embodiments, the composition may comprise a first construct and a second construct as described herein. In embodiments, the composition may comprise a first construct, a second construct, and one or more intervening constructs as described herein. The compositions may comprise a buffer, e.g. phosphate buffered saline (PBS), and any other salts, vitamins, nutrients, carbohydrates, amino acids, fats, and cellular proteins needed to maintain the structural integrity of the viral particles. The compositions may include a single rAAV with a single transgene. The compositions may include two or more rAAVs that when combined and introduced into a single cell are able to concatemerized and allow for transcription, translation and functional expression of the transgene in the form of a functional protein. The compositions may include a pharmaceutically acceptable carrier.

### Method for Delivering a Transgene:

In a ninth aspect, the present disclosure provides methods of delivering a transgene to a subject. The methods comprise administering a composition of rAAV virus particles described herein to the subject. “Delivering a transgene” refers to methods that result in transgene expression in one or more of the subject's cells.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. In some embodiments, the virus particle is administered by vascular injection.

The virus particle may be administered with a pharmaceutically acceptable carrier. “Pharmaceutically acceptable carriers” are known in the art and include, but are not limited to, diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

Ideally, the virus particles are administered in a therapeutically effective amount. The term “therapeutically effective amount” refers to an amount sufficient to effect beneficial or desirable biological or clinical results. Methods for determining an effective means of administration and dosage are well known to those of skill in the art and will vary with the formulation used for therapy and the subject (e.g., species, age, health, etc.) being treated. Single or multiple administrations can be carried out with the dose level and pattern being selected by the treating physician. In some embodiments, the virus particle is administered at a dose of 1×1012 viral genome/kg (vg/kg) or less.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex. Preferably, the subject is a human.

Transgene expression can be detected using any suitable method known in the art. For example, when the transgene encodes a protein, the protein product may be detected using an enzyme-linked immunoassay (ELISA), dot blot, western blot, flow cytometry, mass spectrometry, or chromatographic method. When the transgene encodes a functional RNA, the RNA product may be detected using reverse transcription and polymerase chain reaction (RT-PCR) or Northern blotting.

In a tenth aspect, the present disclosure provides methods of delivering a transgene to a cell, the method comprising contacting the cell with a composition of rAAV virus particles described. As used herein, the term “contacting” includes contacting cells directly or indirectly in vivo, in vitro, or ex vivo. Further, contacting a cell includes adding an rAAV composition to a cell culture.

It should be apparent to those skilled in the art that many additional modifications besides those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The term “substantial identity” of polynucleotide sequences means that a polynucleotide comprises a sequence that has at least 85%, and up to 100%, sequence identity to the SEQ ID. Percent identity may be any integer from 85% to 100%. More preferred embodiments include at least: 85%, 86%, 87%, 88%, 89%, 90%, 91%, 92%, 93%, 94%, 95%, 96%, 97%, 98% or 99% compared to a reference sequence using a sequence alignment program; preferably BLAST using standard parameters. These values can be appropriately adjusted to determine corresponding identity of proteins encoded by two nucleotide sequences by taking into account codon degeneracy, amino acid similarity, reading frame positioning, and the like.

“Substantial identity” of amino acid sequences for purposes of this invention normally means polypeptide sequence identity of at least 85%. Preferred percent identity of polypeptides can be any integer from 85% to 100%. More preferred embodiments include at least 85%, 86%, 87%, 88%, 89%, 90%, 91%, 92%, 93%, 94%, 95%, 96%, 97%, 98%, or 99% to a reference sequence using a sequence alignment program; preferably BLAST using standard parameters.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### Examples

In the following Example, the inventors identify recombined junction sequences that arise from AAV2 rearrangement into concatamers.

Adeno-Associated Viruses Type 2 (AAV2) are single-stranded DNA viruses of the parvovirus family that are 4.7 kilobases (kb) long and contain Inverted Terminal Repeats (ITRs) at either end. These ITRs serve as cis-elements that regulate virus replication and packaging. AAV2 genomes have been modified by replacing the intervening genome between the ITRs with transgenes to engineer recombinant AAV2 (rAAV) gene therapy vectors, as illustrated in FIG. 1. These rAAV gene therapy vectors have become the vectors of choice for treating genetic diseases. Several FDA approved therapeutics that perform these functions are currently in use for the treatment of spinal muscular atrophy, Duchenne muscular dystrophy, hemophilia and CAR T cells. However, due to limitations in the packaging capacity of the AAV2/rAAV capsid, the size of the rAAV transgene that can be accommodated inside the vector capsid is limited to 5 kb. This has proven to be a limitation in rAAV gene therapy vector technology, preventing the use of rAAV transgenes that are larger than 5 kb in size, and thereby limiting the widespread application of rAAV-based gene therapies.

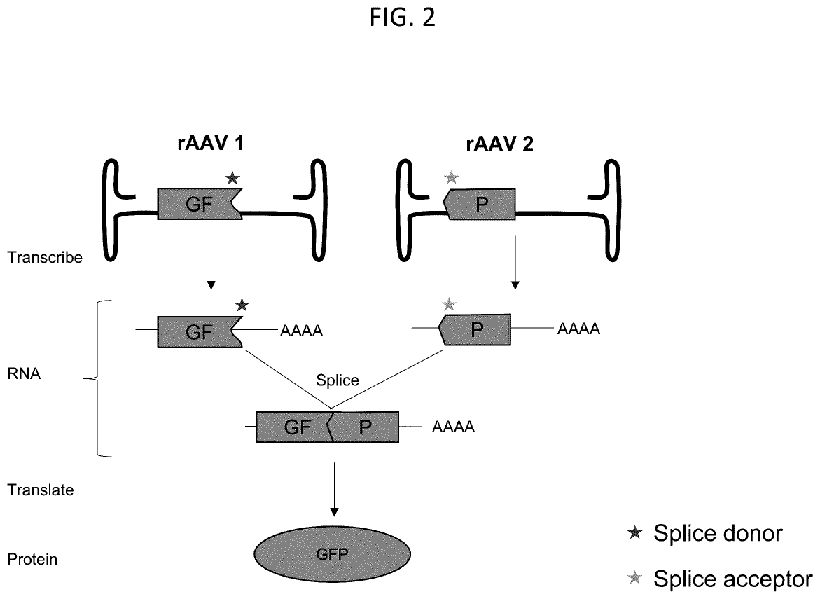

The current state of the art in using smaller rAAV genomes to reconstitute larger transgenes leverages the principles of RNA processing from multiple independent rAAV vectors, as illustrated in FIG. 2. Splice donor and acceptor sites are engineered into the relevant regions of the vectors so that the primary transcripts undergo post-transcriptional processing to facilitate formation of large transcripts that encode large proteins. However, this technique is predicated on the principle that the RNA transcripts generated by independent rAAV genomes are transduced at an equivalent rate and the independent vectors persist at the same levels long-term.

AAV2 and rAAV genomes recombine together to form extrachromosomal concatemers that serve as an expression platform for the viral genes and therapeutic transgenes, respectively. Upon infection, AAV2 forms head-to-tail multimeric concatemers that remain unintegrated, as illustrated in FIG. 3. Depending on the cell type, about 95% of the genomes multimerize extrachromosomally to form these concatemers and 5% or less integrate into a well characterized integration site in chromosome 19. The inventors sought to better understand AAV rearrangement and leverage the biology of concatemer formation to design rAAV vectors that can be engineered to rearrange to form large transgenes.

### Identification of the AAV2 Recombinant Junction

The inventors hypothesized that if two linear AAV2 genomes rearrange in head-to-tail orientation, then PCR primers that emanate outwards (forward primer complementary to the 3′ end and a reverse primer complementary to the 5′ end on the minus strand) will amplify the AAV2 rearrangement.

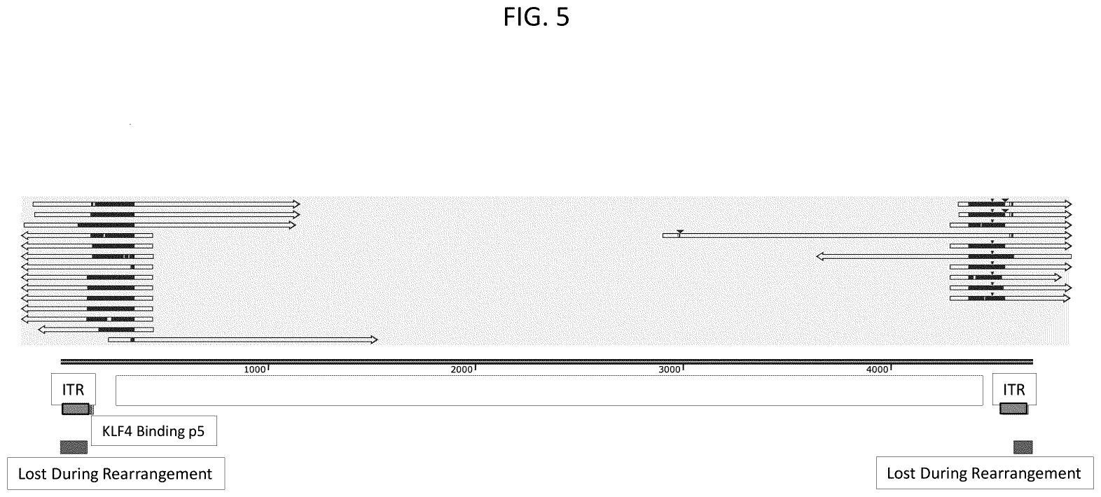

Human Embryonic Kidney (HEK-293T) cells were transduced with AAV2 for 24 hours before being harvested for genomic DNA extraction. To identify the AAV2 recombination junctions, the inventors designed primers that are complementary to the 5′ and 3′ ends of the AAV2 genomes but amplify DNA in the outward orientation (schematized in FIG. 4). These PCR products were amplified using Taq polymerase to add Adenine overhangs to the amplicon terminals. These gel-purified PCR products were ligated into a vector backbone using the Topo-TA-cloning kit, transformed into competent cells (Stellar cells, Takara Biosciences) and screened for Ampicillin resistance. Colonies were selected for miniprep cultures and submitted for sequencing using M13 and T7 sequencing primers complementary to the pCR-TOPO plasmid backbone. These sequencing results yielded multiple hybrid junction sequences generated from two successive AAV genomes (shown in FIG. 5 as arrows). For further validation studies, the inventors focused on concatemer pairs where the 3′ AAV2's 144 bp site rearranges with the 4554 bp site of the 5′ AAV2 in linear orientation. The intervening AAV2 sequence is lost (labelled as “Lost during rearrangement” in FIG. 5).

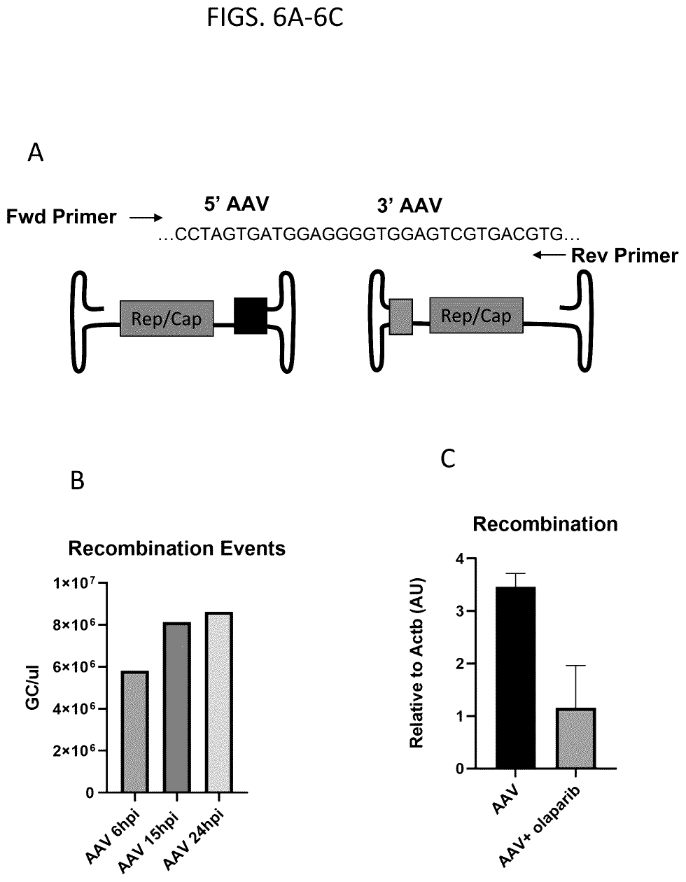

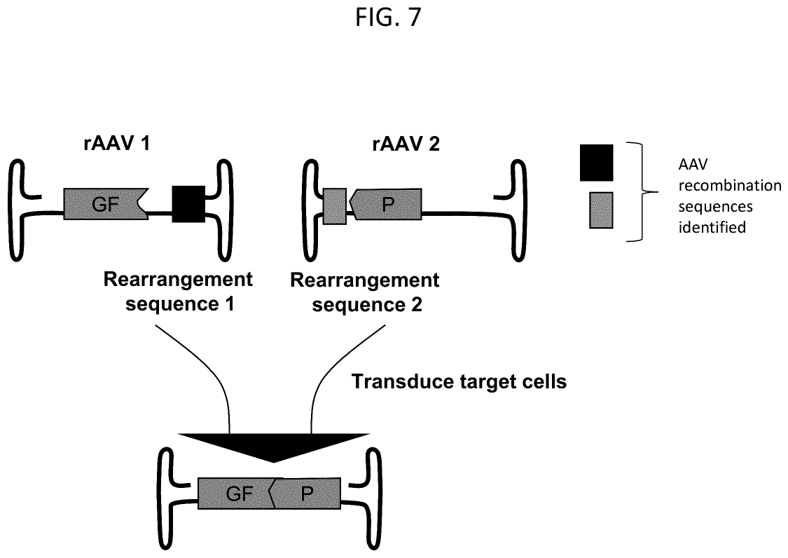

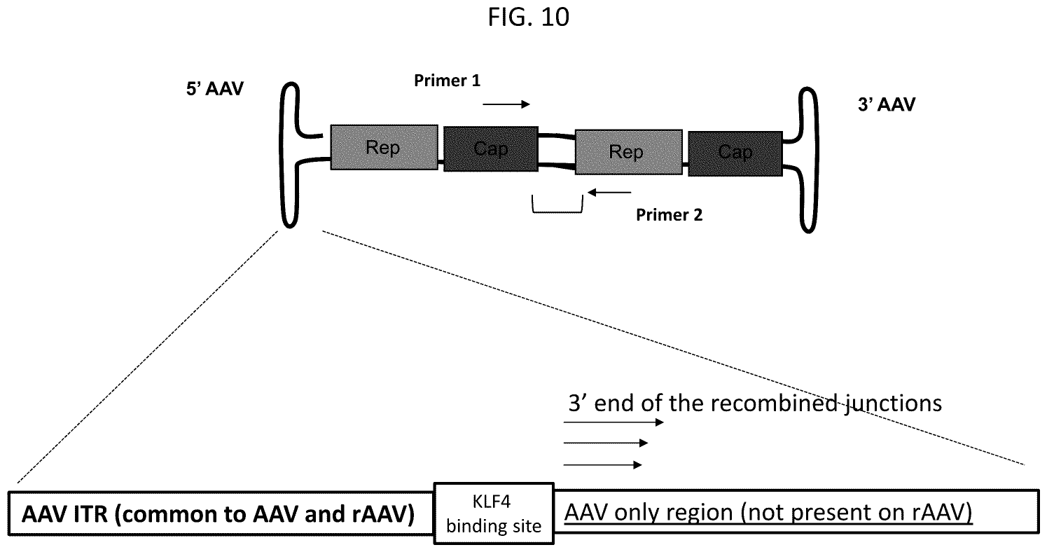

Building on these findings, the inventors designed qPCR primers that can detect these hybrid junctions (FIG. 6A). The positioning of the primers is further illustrated in FIG. 10. During a time-course of AAV2 infection in HEK293T cells, the inventors discovered that the formation of these AAV2 hybrid junctions increase from 6 hours post infection (hpi) to 15 hpi and to 24 hpi (FIG. 6B). It was hypothesized that the AAV2 rearrangement is carried out by cellular DNA damage response (DDR) machinery. To determine whether the cellular DDR regulates AAV2 concatemer formation, the inventors treated AAV2-transduced 293T cells with Olaparib, an inhibitor of the DNA repair protein PARP1. The rearrangement frequencies diminished in Olaparib-treated cells (FIG. 6C), implicating the cellular DNA repair pathway in AAV2 recombination. Taken together, these studies are the first to identify the hybrid junctions that make up linear AAV2 concatemers and the host signaling pathways that regulate rearrangements, which can be usurped for engineering rAAV recombination. For example, the sequences needed to yield these recombined junction sequences may be used to express large transgenes via multiple rAAV2 constructs, as illustrated in FIG. 7.

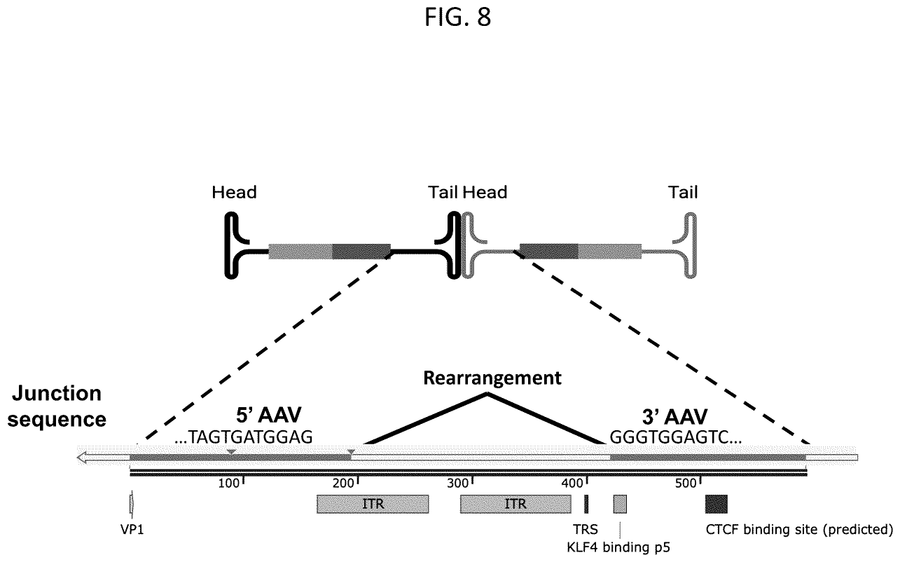

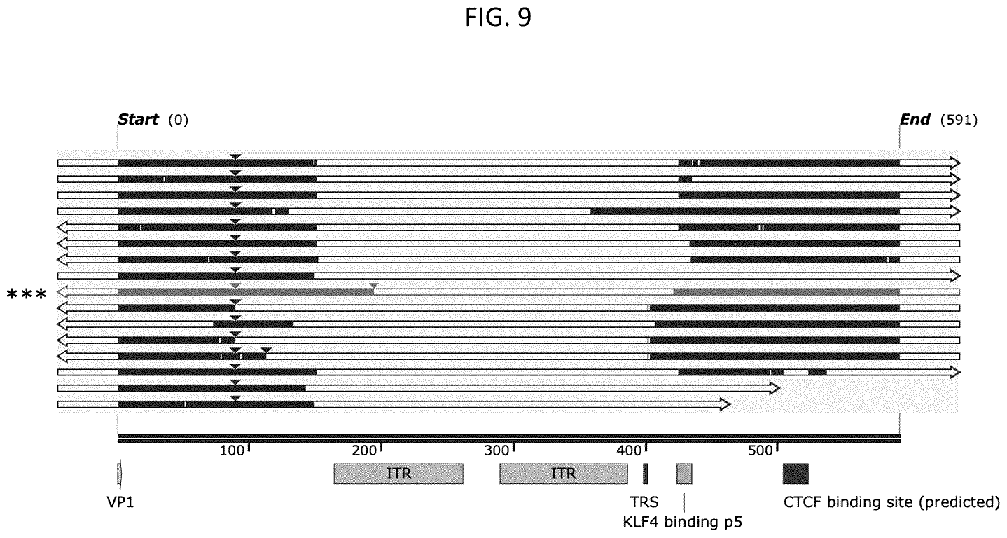

The inventors developed a reference genome for the ideal linear head-to-tail rearrangement of two AAV monomers with the 3′ end of the first AAV recombining with the 5′ end of the second AAV, shown in FIG. 8. The high-throughput sequencing data of AAV rearrangements was aligned to this recombination and illustrated in FIG. 9. Additional recombined junction sequences identified were identified (SEQ ID NOs: 11-21, where N is any nucleotide).

FIG. 10 illustrates regions of the 5′ end of the AAV that the inventors have identified as being involved in recombination. In traditional rAAV, only the ITR regions are retained, and the remaining AAV genome is removed. As shown herein, the KLF4 binding site is important for recombination and portions of the AAV only region are present in AAV dimers after recombination.

### SEQUENCES

TABLE 1
Sequences

| SEQ | | |
|---|---|---|
| ID | | |
| NO: | Description | Sequence |
| 1 | 5′ AAV | GGGTGGAGTCGTGACGTG |
| | recombined | |
| | junction sequence | |
| 2 | 3′ AAV | CCTAGTGATGGAG |
| | recombined | |
| | junction sequence | |
| 3 | AAV2 | ttggccactccctctctgegcgctcgctcgctcactgaggccgggcgaccaaaggtcgcccgacgcccgggctttgcccgg |
| | Genome | gcggcctcagtgagcgagcgagcgcgcagagagggagtggccaactccatcactaggggttcctggaggggtggagtcg |
| | | tgacgtgaattacgtcatagggttagggaggtcctgtattagaggtcacgtgagtgttttgcgacattttgcgacaccatgtggt |
| | | cacgctgggtatttaagcccgagtgagcacgcagggtctccattttgaagcgggaggtttgaacgcgcagccgccatgccgg |
| | | ggttttacgagattgtgattaaggtccccagcgaccttgacgagcatctgcccggcatttctgacagctttgtgaactgggtggc |
| | | cgagaaggaatgggagttgccgccagattctgacatggatctgaatctgattgagcaggcacccctgaccgtggccgagaa |
| | | gctgcagcgcgactttctgacggaatggcgccgtgtgagtaaggccccggaggcccttttctttgtgcaatttgagaagggag |
| | | agagctacttccacatgcacgtgctcgtggaaaccaccggggtgaaatccatggttttgggacgtttcctgagtcagattcgcg |
| | | aaaaactgattcagagaatttaccgcgggatcgagccgactttgccaaactggttcgcggtcacaaagaccagaaatggcgc |
| | | cggaggcgggaacaaggtggtggatgagtgctacatccccaattacttgctccccaaaacccagcctgagctccagtgggc |
| | | gtggactaatatggaacagtatttaagcgcctgtttgaatctcacggagcgtaaacggttggtggcgcagcatctgacgcacgt |
| | | gtcgcagacgcaggagcagaacaaagagaatcagaatcccaattctgatgcgccggtgatcagatcaaaaacttcagccag |
| | | gtacatggagctggtcgggtggctcgtggacaaggggattacctcggagaagcagtggatccaggaggaccaggcctcat |
| | | acatctccttcaatgcggcctccaactcgcggtcccaaatcaaggctgccttggacaatgcgggaaagattatgagcctgact |
| | | aaaaccgcccccgactacctggtgggccagcagcccgtggaggacatttccagcaatcggatttataaaattttggaactaaa |
| | | cgggtacgatccccaatatgcggcttccgtctttctgggatgggccacgaaaaagttcggcaagaggaacaccatctggctgt |
| | | ttgggcctgcaactaccgggaagaccaacatcgcggaggccatagcccacactgtgcccttctacgggtgcgtaaactgga |
| | | ccaatgagaactttcccttcaacgactgtgtcgacaagatggtgatctggtgggaggaggggaagatgaccgccaaggtcgt |
| | | ggagtcggccaaagccattctcggaggaagcaaggtgcgcgtggaccagaaatgcaagtcctcggcccagatagacccga |
| | | ctcccgtgatcgtcacctccaacaccaacatgtgcgccgtgattgacgggaactcaacgaccttcgaacaccagcagccgtt |
| | | gcaagaccggatgttcaaatttgaactcacccgccgtctggatcatgactttgggaaggtcaccaagcaggaagtcaaagact |
| | | ttttccggtgggcaaaggatcacgtggttgaggtggagcatgaattctacgtcaaaaagggtggagccaagaaaagacccgc |
| | | ccccagtgacgcagatataagtgagcccaaacgggtgcgcgagtcagttgcgcagccatcgacgtcagacgcggaagctt |
| | | cgatcaactacgcagacaggtaccaaaacaaatgttctcgtcacgtgggcatgaatctgatgctgtttccctgcagacaatgcg |
| | | agagaatgaatcagaattcaaatatctgcttcactcacggacagaaagactgtttagagtgctttcccgtgtcagaatctcaacc |
| | | cgtttctgtcgtcaaaaaggcgtatcagaaactgtgctacattcatcatatcatgggaaaggtgccagacgcttgcactgcctgc |
| | | gatctggtcaatgtggatttggatgactgcatctttgaacaataaatgatttaaatcaggtatggctgccgatggttatcttccagat |
| | | tggctcgaggacactctctctgaaggaataagacagtggtggaagctcaaacctggcccaccaccaccaaagcccgcagag |
| | | cggcataaggacgacagcaggggtcttgtgcttcctgggtacaagtacctcggacccttcaacggactcgacaagggagag |
| | | ccggtcaacgaggcagacgccgcggccctcgagcacgacaaagcctacgaccggcagctcgacagcggagacaacccg |
| | | tacctcaagtacaaccacgccgacgcggagtttcaggagcgccttaaagaagatacgtcttttgggggcaacctcggacgag |
| | | cagtcttccaggcgaaaaagagggttcttgaacctctgggcctggttgaggaacctgttaagacggctccgggaaaaaagag |
| | | gccggtagagcactctcctgtggagccagactcctcctcgggaaccggaaaggcgggccagcagcctgcaagaaaaagat |
| | | tgaattttggtcagactggagacgcagactcagtacctgacccccagcctctcggacagccaccagcagccccctctggtctg |
| | | ggaactaatacgatggctacaggcagtggcgcaccaatggcagacaataacgagggcgccgacggagtgggtaattcctc |
| | | gggaaattggcattgcgattccacatggatgggcgacagagtcatcaccaccagcacccgaacctgggccctgcccaccta |
| | | caacaaccacctctacaaacaaatttccagccaatcaggagcctcgaacgacaatcactactttggctacagcaccccttggg |
| | | ggtattttgacttcaacagattccactgccacttttcaccacgtgactggcaaagactcatcaacaacaactggggattccgacc |
| | | caagagactcaacttcaagctctttaacattcaagtcaaagaggtcacgcagaatgacggtacgacgacgattgccaataacc |
| | | ttaccagcacggttcaggtgtttactgactcggagtaccagctcccgtacgtcctcggctcggcgcatcaaggatgcctcccg |
| | | ccgttcccagcagacgtcttcatggtgccacagtatggatacctcaccctgaacaacgggagtcaggcagtaggacgctcttc |
| | | attttactgcctggagtactttccttctcagatgctgcgtaccggaaacaactttaccttcagctacacttttgaggacgttcctttcc |
| | | acagcagctacgctcacagccagagtctggaccgtctcatgaatcctctcatcgaccagtacctgtattacttgagcagaacaa |
| | | acactccaagtggaaccaccacgcagtcaaggcttcagttttctcaggccggagcgagtgacattcgggaccagtctaggaa |
| | | ctggcttcctggaccctgttaccgccagcagcgagtatcaaagacatctgcggataacaacaacagtgaatactcgtggactg |
| | | gagctaccaagtaccacctcaatggcagagactctctggtgaatccgggcccggccatggcaagccacaaggacgatgaa |
| | | gaaaagttttttcctcagagcggggttctcatctttgggaagcaaggctcagagaaaacaaatgtggacattgaaaaggtcatg |
| | | attacagacgaagaggaaatcaggacaaccaatcccgtggctacggagcagtatggttctgtatctaccaacctccagagag |
| | | gcaacagacaagcagctaccgcagatgtcaacacacaaggcgttcttccaggcatggtctggcaggacagagatgtgtacct |
| | | tcaggggcccatctgggcaaagattccacacacggacggacattttcacccctctcccctcatgggggattcggacttaaac |
| | | accctcctccacagattctcatcaagaacaccccggtacctgcgaatccttcgaccaccttcagtgcggcaaagtttgcttcctt |
| | | catcacacagtactccacgggacaggtcagcgtggagatcgagtgggagctgcagaaggaaaacagcaaacgctggaatc |
| | | ccgaaattcagtacacttccaactacaacaagtctgttaatgtggactttactgtggacactaatggcgtgtattcagagcctcgc |
| | | cccattggcaccagatacctgactcgtaatctgtaattgcttgttaatcaataaaccgtttaattcgtttcagttgaactttggtctctg |
| | | cgtatttctttcttatctagtttccatggctacgtagataagtagcatggcgggttaatcattaactacaaggaacccctagtgatg |
| | | gagttggccactccctctctgcgcgctcgctcgctcactgaggccgggcgaccaaaggtcgcccgacgcccgggetttgcc |
| | | cgggcggcctcagtgagcgagcgagcgcgcagagagggagtggccaa |
| 4 | Recombined | CCTAGTGATGGAGGGGTGGAGTCGTGACGTG |
| | junction | |
| 5 | KLF4 binding | aggggtggagtc |
| | site | |
| 6 | 5′ recombinant | ggaggggtggagtcgtgacgtgaattacgtcatagggttagggaggtcctgtattagaggtcacgtgagtgttttgcgacatttt |
| | junction region | gcgacaccatgtggtcacgctgggtatttaagcccgagtgagcacgcagggtctccattttgaagcgggaggtttgaacgcg |
| | | cagccgccatgccggggttttacgagattgtga |
| 7 | 3′ recombinant | cccattggcaccagatacctgactcgtaatctgtaattgcttgttaatcaataaaccgtttaattcgtttcagttgaactttggtctctg |
| | junction region | cgtatttctttcttatctagtttccatggctacgtagataagtagcatggcgggttaatcattaactacaaggaacccctagtgatgg |
| | (includes a | agttggccactccctctctgegcgctc |
| | portion of the | |
| | 3′ ITR) | |
| 8 | 5′ ITR and 5′ | ttggccactccctctctgcgcgctcgctcgctcactgaggccgggcgaccaaaggtcgcccgacgcccgggctttgcccgg |
| | recombinant | gcggcctcagtgagcgagcgagcgcgcagagagggagtggccaactccatcactaggggttcctggaggggtggagtcg |
| | junction region | tgacgtgaattacgtcatagggttagggaggtcctgtattagaggtcacgtgagtgttttgcgacattttgcgacaccatgtggtc |
| | | acgctgggtatttaagcccgagtgagcacgcagggtctccattttgaagcgggaggtttgaacgcgcagccgccatgccgg |
| | | ggttttacgagattgtga |
| 9 | 3′ recombinant | cccattggcaccagatacctgactcgtaatctgtaattgcttgttaatcaataaaccgtttaattcgtttcagttgaactttggtctctg |
| | junction region | cgtatttctttcttatctagtttccatggctacgtagataagtagcatggcgggttaatcattaactacaaggaacccctagtgatgg |
| | and 3′ ITR | agttggccactccctctctgegcgctcgctcgctcactgaggccgggcgaccaaaggtcgcccgacgcccgggctttgccc |
| | | gggcggcctcagtgagcgagcgagcgcgcagagagggagtggccaa |
| 10 | 5′ recombinant | ctctgcgcgctcgctcgctcactgaggccgggcgaccaaaggtcgcccgacgcccgggetttgcccgggcggcctca |
| | junction region | gtgagcgagcgagcgcgcagagagggagtggccaactccatcactaggggttcctggaggggtggagtcgtgacgtg |
| | (FIG. 10) | aattacgtcatagggttagggaggtcctgtattagaggtcacgtgagtgttttgcgacattttgcgacaccatgtggtcacgctg |
| | AAV ITR bolded | ggtatttaag |
| | AAV only | |
| | region underlined | |
| 11 | Recombined | CCCCTAGTGATGNAGGGGTGGAGTCNTGACNT |
| | Junction #2 | |
| | (5′ sequence | |
| | bolded) | |
| 12 | Recombined | CCTAGTGATGGAGGGGTGGAGTCNTGA |
| | Junction #3 | |
| | (5′ sequence | |
| | bolded) | |
| 13 | Recombined | TAGTGATGGAGGGGTGGAGTCGTGACG |
| | Junction #4 | |
| | (5′ sequence | |
| | bolded) | |
| 14 | Recombined | CATGGCGGGTTAGTCATTAACTACCGGCCTCAGTGA |
| | Junction #5 | |
| | (5′ sequence | |
| | bolded) | |
| 15 | Recombined | AAGGAACCCCTAGTGATGGAGTCGTGACGTGAATTACGTCATAGGGTT |
| | Junction #6 | |
| | (5′ sequence | |
| | bolded) | |
| 16 | Recombined | AGGAACCCCTAGTGATGGAGGGGTGGAGTCGTGACGTG |
| | Junction #7 | |
| | (5′ sequence | |
| | bolded) | |
| 17 | Recombined | AACCCCTAGTGATGGAGTCGTGACGTGAATTACGTCATAGG |
| | Junction #8 | |
| | (5′ sequence | |
| | bolded) | |
| 18 | Recombined | CTGAGGCCGCCCGGGCGGAGGGGTGGAGTCGT |
| | Junction #9 | |
| | (5′ sequence | |
| | bolded) | |
| 19 | Recombined | ATTTCTTTCTTATCTAGTTTCCATGCTNTAGNNCANGGCTANGTAG |
| | Junction #10 | |
| | (5′ sequence | |
| | bolded) | |
| 20 | Recombined | TTAATCATTAACTACAAGCACTAGGGGTTCCT |
| | Junction #11 | |
| | (5′ sequence | |
| | bolded) | |
| 21 | Recombined | ACCCCTAGTGATGGAGGGGTGGAGTCGTGACGTGAATT |
| | Junction #12 | |
| | (5′ sequence | |
| | bolded) | |

1. A construct for producing a recombinant adeno-associated virus (rAAV) vector, the construct comprising from 5′ to 3′: a 5′ inverted terminal repeat (ITR), a first recombinant junction sequence, a promoter operably linked to at least a portion of a transgene, and a 3′ ITR.

2. The construct of claim 1, wherein the first recombinant junction sequence comprises a KLF4 binding site comprising SEQ ID NO: 5.

3. The construct of claim 2, wherein the first recombinant junction sequence consists of the KLF4 binding site.

4. The construct of claim 1, wherein the first recombinant junction sequence comprises SEQ ID NO: 6 or a portion thereof.

5. (canceled)

6. The construct of claim 1, further comprising a second recombinant junction sequence, wherein the second recombinant junction sequence is between the transgene and the 3′ ITR and may overlap with the 3′ ITR.

7. The construct of claim 6, wherein the second recombinant junction sequence comprises at least the last 45 nucleotides of SEQ ID NO: 7.

8. (canceled)

9. An rAAV vector comprising the construct of claim 1.

10. (canceled)

11. A composition comprising an rAAV vector, the rAAV vector comprising the construct of claim 1.

12. A method of delivering a transgene to a cell comprising contacting the cell with the composition of claim 11.

13. A method of delivering a transgene to a subject, the method comprising: administering to the subject, the composition of claim 11.

14. A system for expressing a transgene, the system comprising:
a first construct for producing a first recombinant adeno-associated virus (rAAV) vector comprising, from 5′ to 3′: a 5′ inverted terminal repeat (ITR), a promoter operably linked to a 5′ portion of the transgene, a second recombinant junction sequence, and a 3′ ITR, wherein the second recombinant junction sequence is between the transgene and the 3′ ITR and may overlap with the 3′ ITR; and
a second construct for producing a second rAAV vector comprising, from 5′ to 3′: the 5′ ITR, a first recombinant junction sequence, a 3′ portion of the transgene, a polyadenylation site, and the 3′ ITR.

15. The system of claim 14, wherein the first construct does not comprise the first recombinant junction sequence, and wherein the second construct does not comprise the second recombinant junction sequence.

16. The system of claim 14, wherein the first recombinant junction sequence comprises a KLF4 binding site comprising SEQ ID NO: 5.

17. (canceled)

18. The system of claim 14, wherein the first recombinant junction sequence comprises SEQ ID NO: 6 or a portion thereof.

19-21. (canceled)

22. The system of claim 21, wherein the 5′ portion of the transgene and the 3′ portion of the transgene each comprise between about 200 nucleotides and about 4000 nucleotides.

23. The system of claim 14, wherein the first construct further comprises a splice donor site at the 3′ end of the 5′ portion of the transgene and the second construct further comprises a splice acceptor site and a branch site at the 5′ end of the 3′ portion of the transgene.

24. A recombinant AAV (rAAV) vector comprising the first construct or the second construct of claim 14.

25. (canceled)

26. A composition comprising a first rAAV particle comprising the first construct and a second rAAV particle comprising the second construct of claim 14.

27. (canceled)

28. A method of delivering a transgene to a subject, the method comprising administering to the subject the composition of claim 26.

29. A system for expressing a transgene, the system comprising:
a first construct for producing a first recombinant adeno-associated virus (rAAV) vector, the first construct comprising, from 5′ to 3′: a 5′ ITR, a promoter operably linked to a 5′ portion of the transgene, a first 3′ recombinant junction sequence, and a 3′ ITR;
a second construct for producing a second rAAV vector, the second construct comprising, from 5′ to 3′: the 5′ ITR, a second 5′ recombinant junction sequence, a 3′ portion of the transgene, a polyadenylation site, and the 3′ ITR; and
an intervening construct for producing an intervening rAAV vector, the intervening construct comprising, from 5′ to 3′: the 5′ ITR, a first 5′ recombinant junction sequence, an internal portion of the transgene, a second 3′ recombinant junction sequence, and the 3′ ITR; wherein the first 5′ recombinant junction sequence and the second 5′ recombination sequence are different;
wherein the first 3′ recombinant junction sequence and the second 3′ recombinant junction sequence are different;
wherein the first 5′ recombinant junction sequence and the first 3′ recombinant junction sequence are such that when recombined, the 5′ portion of the transgene is in frame with the internal portion of the transgene; and
wherein the second 3′ recombinant junction sequence and the second 5′ recombinant junction sequence are such that when recombined, the internal portion of the transgene is in frame with the 3′ portion of the transgene.

30-33. (canceled)
